(19) United States
(12) Patent Application Publication
(10) Pub. No.: US 2018/0327833 A1
(43) Pub. Date: Nov. 15, 2018

(54) BIOFUNCTIONALIZED NANOSHELL IMMOBILIZED MICROARRAYS AND APPLICATIONS THEREOF

(71) Applicant: ARIZONA BOARD OF REGENTS ON BEHALF OF THE UNIVERSITY OF ARIZONA, Tucson, AZ (US)

(72) Inventors: Craig A. Aspinwall, Tucson, AZ (US); Phuong-Diem Nguyen, Tucson, AZ (US); Jinyan Wang, Tucson, AZ (US); Xuemin Wang, Tucson, AZ (US)

(21) Appl. No.: 15/979,185

(22) Filed: May 14, 2018

Related U.S. Application Data
(60) Provisional application No. 62/505,720, filed on May 12, 2017.

(51) Int. Cl. C12Q 1/6874; C12Q 1/6809; C12Q 1/6816; C12Q 1/6832; C12Q 1/6837

(52) U.S. Cl. CPC C12Q 1/6874; C12Q 1/6809; C12Q 1/6837; C12Q 1/6832; C12Q 1/6816

(57) ABSTRACT

Microarray platforms and methods of fabricating said microarrays without traditional high aspect ratio barriers used to define individual array elements are described herein. Self-assembled nanoshells were stabilized with a polymerized scaffold to enhance the stability in physiological conditions and serve as an optical transducer upon molecular recognition events. Soft photolithography combined with surface chemistry was developed for covalent immobilization of nanoshells onto the pre-patterned arrayed microspots for rapid multiplexed detection of membrane-binding analytes. This robust fabrication methodology is amenable for general lipid structures, and thus facilitates the integration of stable membrane architectures into diagnostic and prognostic platforms. In particular, the microarray platform may be used in diverse applications ranging from the detection of pathogens, such bacterial toxin in biological matrices, to cellular membrane studies.

### CROSS REFERENCE

This application claims priority to U.S. Provisional Application No. 62/505,720 filed May 12, 2017, the specification(s) of which is/are incorporated herein in their entirety by reference.

### GOVERNMENT SUPPORT

This invention was made with government support under Grant No. R01 GM116946, awarded by NIH. The government has certain rights in the invention.

### FIELD OF THE INVENTION

The present invention relates to a novel microarray platform and fabrication process for multiplexed detection of a wide range of membrane-interacting particles.

### BACKGROUND OF THE INVENTION

Cell membranes serve as transducers of the extracellular environment into intracellular signaling, a critical role in cell function. A wide range of binding interactions occur at the cell membrane via protein-ligand, protein-protein, and other interactions that are highly selective and highly specific. The capability to utilize these interactions for purification, preconcentration and quantification would provide a key enabling methodology for studies in biology, food safety, environmental chemistry and many other areas, akin to the rapid growth of antibody based methodologies. For example, G-protein coupled receptors and other transmembrane proteins bind a wide range of hormones, neurotransmitters, chemotransducers, pharmaceuticals, and other species with high affinity. In many cases, antibodies are not available for small molecules that exist across species, thus making binding dependent assays difficult to perform. The capability to integrate biological transmembrane protein-ligand interactions, as well as other membrane localized binding events, into array analytical platforms enable a new generation of assays. Furthermore, many toxins, including food-borne bacterial toxins such as cholera toxin, recognize specific components in the cell membrane (GM-1 in the case of cholera toxin) that could be used for enhanced binding and assay capability.

The primary limitation of preparing analytical platforms that utilize membrane-specific binding interactions is the relative instability of the membrane. Phospholipids, one of the principle components in membrane structure, provide an inert supporting surface for the biological interactions of transmembrane proteins to occur easily on the cell membrane. In other words, phospholipids play an important role in maintaining the functions of transmembrane proteins and serve as the key interface for membrane localized binding events. This represents an opportunity to integrate these binding interactions into an array that allows for rapid and simultaneous quantification of multiple particles.

Planar supported lipid bilayer (PSLB) microarrays have been fabricated but have a disadvantage in that these materials yield a ca. 1 nm water layer between the PSLB and the underlying support substrate, which is insufficient for functional integration of many membrane proteins. In addition, traditional micron-sized barriers may limit the diffusion into arrayed vesicles. PSLB microarrays require a specialized surface chemistry to keep the probe molecules and associated lipid membrane confined to the arrayed microspots while maintaining the essential lateral movement. The mechanical stability of the non-covalently attached liposomes can be a very significant issue that can compromise the feasibility of the microarray.

Phospholipid nanoshells (PPN) provide a convenient biomimetic platform that is increasingly used to quantify biophysical and biochemical processes related to cell function. These hollow nano-scale spheres are particularly attractive due to their unique structural and material properties, including the ability to encapsulate compounds in their aqueous core with minimized diffusion restrictions as compared to solid polymeric nanoparticles. The low mechanical stability of self-assembly of lipids presents a significant challenge in using PPNs for many analytical applications; however, there have been several strategies reported for enhancing the stability via integration of polymer supports. A key advantage in this platform lies in the fact that the stabilized PPN architecture can serve as a supporting lipid membrane and provide a multifunctionalized surface for the binding interactions.

In the past decades, microarray techniques have revolutionized studies of gene and protein expression via quantitative and multiplexed analysis of a wide range of desirable targets. PPN microarrays are fundamentally different from DNA and protein microarray due to the size and architecture. Existing array fabrication technologies are not readily amenable to the fabrication of lipid nanoshell microarrays, particularly arrays that lack high aspect ratio, micron-sized barriers. These barriers ultimately limit diffusional access to the nanoshell and significantly enhance nonspecific adsorption that lowers the detection sensitivity.

To further advance the integration of stabilized phospholipid nanoshells, which can be functionalized with a wide array of binding interactions, into a high throughput analysis platform, the present invention provides for a novel nanoshell array patterning platform that is compatible with stabilized phospholipid nanoshells. Further still, the invention resolves the barrier issue by fabricating the PPN microarray without traditional high aspect ratio barriers.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### SUMMARY OF THE INVENTION

It is an objective of the present invention to provide a microarray platform and methods of fabrication thereof, as specified in the claims. Embodiments of the invention are given in the dependent claims. Embodiments of the present invention can be freely combined with each other if they are not mutually exclusive.

According to some aspects, the present invention features a microarray comprising a plurality of microspots and a plurality of vesicles. Each vesicle may be tethered to a microspot of the plurality of microspots via a linker group, which is effective for enhancing mechanical stability of the microarray by covalently immobilizing said vesicle to the microspot. In one aspect, the linker group can have a proximal end comprising a silane moiety attached to a bottom surface of the microspot, and a distal end covalently immobilizing said vesicle to the microspot. Preferably, a height of the boundary and the surface layer surrounding the microspot is less than 10% of a combined height of the vesicle and linker group. In another aspect, the microarray may further comprise a surface layer in which the microspots are formed therein. Without wishing to limit the present invention, the surface layer is effective for minimizing non-specific surface binding outside of the microspots. Each microspot can have a boundary formed by the surface layer.

In other aspects, the present invention provides a microarray platform comprising a plurality of microspots modified with linker groups. Each linker group may have a proximal end comprising a silane moiety attached to a bottom surface of the microspot, and a distal end comprising a reactive functional group. Preferably, the linker groups are effective for enhancing mechanical stability of the microarray platform when said linker groups covalently immobilize vesicles to the microspots. In some embodiments, the microarray platform may further comprise a surface layer in which the microspots are disposed therein. Each microspot can have a boundary formed by the surface layer such that an aspect ratio of the microspot is less than 0.01. Preferably, the surface layer may be effective for minimizing non-specific surface binding outside of the microspots.

In further aspects, the present invention features a method of preparing a microarray. The method may comprise forming an array of microspots and tethering a vesicle to an interior of the microspots via a linker group, thereby enhancing mechanical stability of the microarray by covalently immobilizing said vesicle to the microspot. Preferably, the linker group has a proximal end comprising a silane moiety attached to a bottom surface of the microspot, and a distal end comprising a reactive functional group that reacts with the vesicle. In some embodiments, the array of microspots may be formed in a surface layer that is effective for minimizing non-specific surface binding outside of the microspots. The microspot may have a boundary formed by the surface layer. Preferably, a height of the boundary and the surface layer surrounding the microspot is less than 10% of a combined height of the vesicle and linker.

In some embodiments, the vesicles may comprise a lipid bilayer of lipid monomers. Examples of the lipid monomers include, but are not limited to, polymerizable lipid monomers, non-polymerizable lipid monomers, functionalized lipid monomers, or a combination thereof. In other embodiments, the vesicles may comprise non-lipid monomers. The vesicles may be functionalized with receptors covalently attached to an outer surface of the vesicles or embedded into a lipid structure of the vesicles. Non-limiting examples of the receptors include membrane protein receptors, growth factor receptors, G-protein coupled receptors, ion channels, lipid-derived receptors, glycoprotein receptors, glycolipids, phospholipids, or a combination thereof. In other embodiments, the vesicles may further comprise a polymer scaffold for stabilizing the vesicles.

Without wishing to be bound by a particular theory or mechanism, the present invention describes a novel microarray fabrication approach where the microarray is fabricated without traditional high aspect ratio barriers used to define individual array elements. Additionally, the microarray may be functionalized with stabilized vesicles for rapid, multiplexed detection of membrane-binding analytes. The novel microarray fabrication methodology described herein is amenable to any lipid functionalized or membrane protein-functionalized nanoshells, thus enabling new membrane specific binding elements to be utilized for array formation, but is also sufficiently general to be used in a range of other array applications. In further embodiments, the assay platform of the present invention can enable highly specific detection of analytes via binding to membrane proteins and membrane associated binding events, and detecting the bindings using techniques such as fluorescent imaging or mass spectrometry. As will be described herein, this concept is demonstrated with ganglioside functionalized, stabilized lipid membranes immobilized into an array format for detecting the binding of toxins by fluorescent imaging.

The capability of detecting analyte molecules in solution using highly specific binding events that occur in or at a lipid membrane provides a key innovation for analysis and purification of important species in complex matrices. Integration of such binding interactions into a multiplexed, array format increases the throughput of membrane-functionalized analytical platforms and allows multiple analytes to be characterized simultaneously and/or rapid calibration and quantification.

To the inventors' knowledge, microarrays with stabilized vesicles that are covalently immobilized are novel platforms that have not existed prior to this present invention. Another of the unique and inventive technical feature of the present invention is the use of photolithography for assay development. Without wishing to limit the invention to a particular theory or mechanism, this approach advantageously provides for microarrays without the traditional high aspect ratio barriers, thereby allowing for the immobilized vesicles to be completely accessible to the analytes, while also reducing nonspecific adsorption. Again, to the inventors' knowledge, there have been no microarrays that greatly enhance the capability of the array platform and minimize mass transport limitations due to diffusion. Other advantages of the present invention over existing technologies include, but are not limited to, high membrane stability and flexibility of membrane surface modifications for different applications. None of the presently known prior references or work has the unique inventive technical features of this invention.

### ABBREVIATIONS

AF488: Alexa Fluor 488

BMA: Butylmethacrylate

CTB: Cholera toxin B

DLA: Dynamic light scattering

DOPC: 1,2-dioleoyl-sn-glycero-3-phosphocholine

DSPE-PEG(2000)-NH₂:1,2-distearoyl-sn-glycero-3-phosphoethanolamine-N-[amino(polyethylene glycol)-2000]

EGDMA: Ethylene glycol dimethacrylate

GM1: GM1 ganglioside

PEG: Polyethylene glycol

PPN: Phospholipid nanoshells

SPN: Stabilized phospholipid nanoshell

### BRIEF DESCRIPTION OF THE DRAWINGS

This patent application contains at least one drawing executed in color. Copies of this patent or patent application publication with color drawing(s) will be provided by the Office upon request and payment of the necessary fee.

The features and advantages of the present invention will become apparent from a consideration of the following detailed description presented in connection with the accompanying drawings in which:

FIGS. 1A-1D show a non-limiting schematic for microarray fabrication and cholera toxin detection. In FIG. 1A, pre-functionalized glass slide was generated by treatment of a cleaned glass slide with PEG trimethoxy silane (CH₃(OCH₂CH₂)ₙSi(OCH₃)₃, n=6-9). An AZ3312 photoresist was used to generate 300×300 μm microspot patterns on PEGylated slide. After oxygen plasma etching and photoresist liftoff, patterned slides were treated with epoxy silane and sulfonate derivatives were subsequently introduced. Finally, aminated SPNs were covalently bound to the arrayed slides. FIG. 1B shows a height comparison of the PEG layer and the SPNs bound to the microspot. FIG. 1C shows a non-limiting schematic diagram of a porous SPN nanostructure. SPNs were fabricated with fluorescent dextran encapsulated inside the lipid membrane and functionalized elements, GM1 ganglioside and amine lipid, were embedded inside the lipid bilayer. Following nanoshell formation, a crosslinking agent (EGDMA) and a photoinitiator (BMA) were partitioned into the inner lamellar region of the nanoshells, forming a polymer scaffold to stabilize the lipid structure. FIG. 1D shows a dose-response detection of CTB-AF488 using the fabricated GM1-SPN microarray.

FIGS. 2A-2D show AFM surface characterization of SPN microarray fabrication on a PEG substrate (dashed yellow line indicated the location where a cross-section measurement was sampled). FIG. 2A is a 10×10 μm AFM image of the glass surface after cleaning with MeOH:HCl. FIG. 2B is a 10×10 μm AFM image of the glass surface after grafting with PEG molecules. FIG. 2C is a 10×10 μm AFM image of the hydrophilic glass pattern surrounded with PEG molecules after etching with oxygen plasma in the presence of a photomask. The PEG layer was approximately 1 nm higher than the bare glass surface. FIG. 2D is a 30×30 μm AFM image taken at the edge of a typical patterned microspot on the array, where SPNs were immobilized inside the microspot surrounded by PEG layer, and the difference in height between the SPN immobilized-area and the PEG layer is clearly visible.

FIGS. 3A-3G show characterization of morphology and stability of GM1-SPN on a on a microarray. FIG. 3A shows epifluorescent images of Texas Red dextran doped GM1-SPN taken in PBS buffer, inset picture showed SPN morphology under a transmission electron microscope (TEM) (scale bar 300=nm). The lipid bilayer was stained by uranyl acetate. FIG. 3B shows an intensity-weighted size distribution of unpolymerized and polymerized SPN in the presence of excessive molar ratio of Triton X-100. FIG. 3C shows a size measured by transmission electron microscopy (TEM) picture of polymer scaffold embedded GM1-SPN. FIG. 3D shows fluorescent microscopic characterization of GM1-SPN stability (with and without polymer scaffold) during long-term storage at 4° C. in PBS buffer. Images were taken at the edge of PBS solution on the glass slide. FIG. 3E shows fluorescent retention results of GM1-SPN with and without polymer scaffold during long-term storage at 4° C. in PBS 10 mM, pH 7.4. FIG. 3F is a false color-fluorescent image of Texas Red dextran doped-SPN microarray, taken in PBS buffer, scale bar=1 mm. FIG. 3G is a 1×1 μm AFM image of SPN immobilized in one arrayed microspot, showing preserved spherical structure of SPN immobilized in microarray.

FIGS. 4A-4B show fluorescent images of GM1-SPN microarrays treated with cholera toxins and the corresponding plot during dose-response binding assay. FIG. 4A shows fluorescent images of microarray elements of GM1-SPN microarray treated with a solution of CTB-AF488 at concentrations ranging from 50 ng to 10 μg/mL. FIG. 4B is a plot showing the concentration dependence of total fluorescent signal during dose response-challenge.

FIGS. 5A-5C show fluorescent images of GM1-SPN microarrays treated with cholera toxins and the corresponding plot during competitive binding assay. FIG. 5A shows fluorescent images of microarray elements treated with a fixed concentration of 2.5 μg/mL CTB-AF488 and increasing concentration of non-labeled CTB from 0 to 50 μg/mL. FIG. 5C is a plot showing competitive binding of CTB-AF488 to GM1-SPN microarray by non-labeled CTB. FIG. 5C is a plot of the recovery of fluorescent detection of spiked CTB in shrimp matrices (p<0.05).

### DESCRIPTION OF PREFERRED EMBODIMENTS

Following is a list of elements corresponding to a particular element referred to herein:
100 microarray
110 base substrate
120 surface layer
130 microspot
135 microspot boundary
140 linker group
150 vesicle

When referring to a size or dimension of a structure, the term “nano” refers to sizes ranging from about 0.1 nm to about 900 nm, as understood by one ordinarily skilled in the art. Likewise, the term “micron” refers to sizes ranging from greater than 0.9 μm to about 1000 μm.

As used herein, the term “vesicle” refers to a nano-sized particle. In some embodiments, the vesicle may have a membrane surface enclosing a cavity or sac. The vesicle membrane may comprise lipids, non-lipids, or a combination thereof. In some embodiments, the vesicle may be substantially spherical in size with diameters ranging from about 100 nm to 900 nm, for instance, 100 nm to 500 nm.

As used herein, the term “microspot” refers to a micron-sized surface formed into a substrate. In some embodiments, a boundary of the microspot comprises the substrate. In other embodiments, a bottom surface of the microspot comprises an exposed surface of a base on which the substrate is disposed. As such, the microspot may have the form of a shallow well or pore. In some embodiments, the microspot may be rectangular, square, circular, or oval in shape.

The term “mass transport” in its usual meaning refers to the movement of mass from point to point. As used herein, mass transport can refer to the movement of molecules through barriers or membranes, or the transfer of molecules in a fluid, such as from a fluidic solution to a receptor. Example forms of mass transport include diffusion, convention, and migration. Diffusion limiting mass transport can be caused by structural properties of a system and flow rates and types. It is an objective of the present invention to provide microarray systems that minimizes diffusion limitations, such as dilution of the sample concentration near a surface, so as to facilitate and increase the transport of biological molecules to vesicles of the microarray.

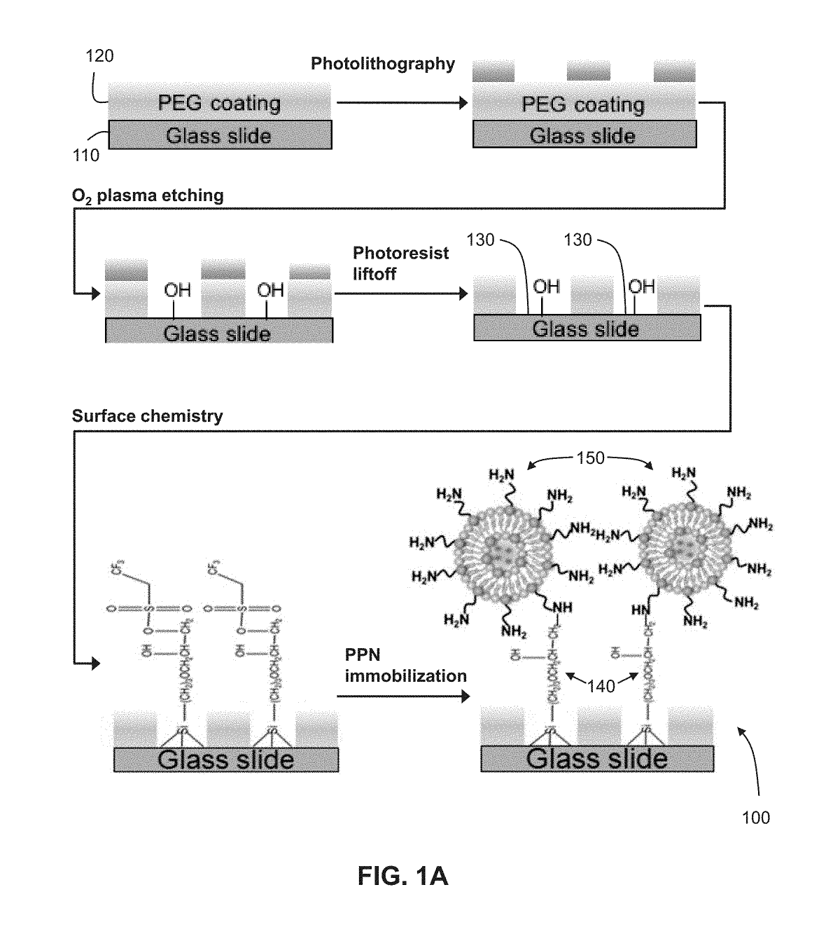

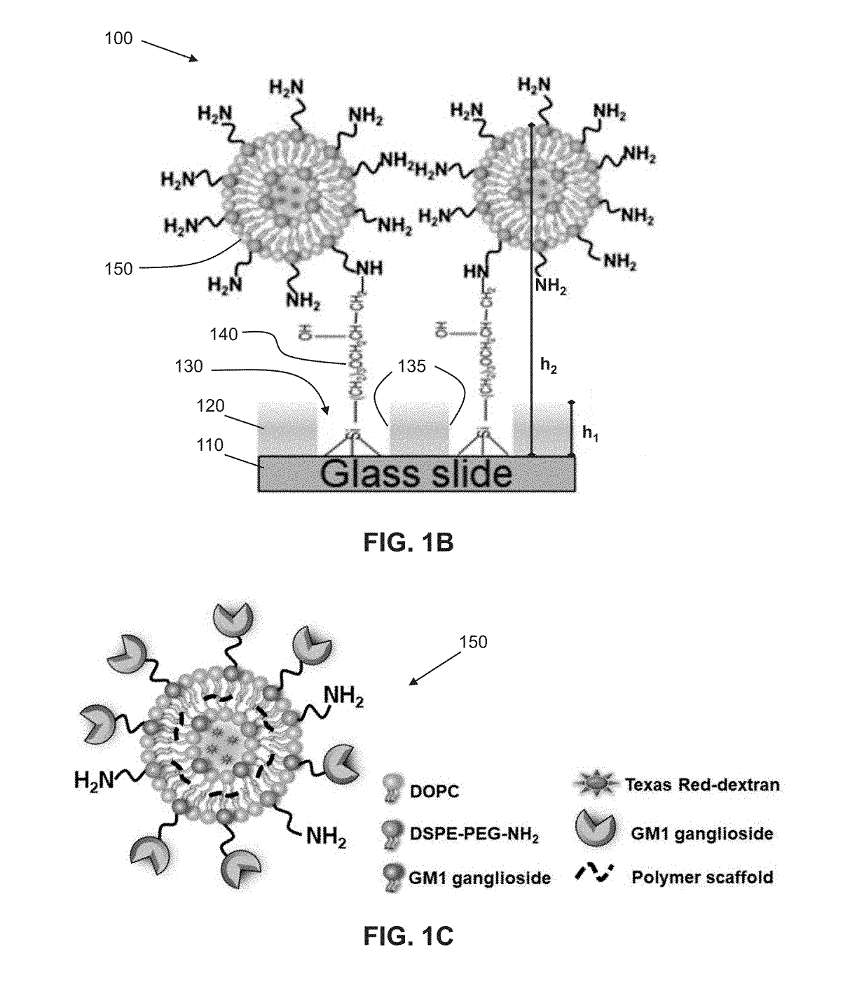

As used herein, the term “mechanical stability” refers to a structural characteristic of the vesicles and microarrays to withstand mechanical forces. The mechanical stability of the vesicles may be increased by connecting their lipid bilayer to a polymer scaffold, thereby preventing the vesicles from being dispersed into the fluid. With respect to the microarray, the mechanical stability may be increased by the use of linker molecules to covalently immobilize the vesicles, thus, the vesicles remain fixed to the microspot while the microarray is being manipulated in fluidic environments, as shown in FIG. 1D.

As known to one of ordinary skill in the art, a “contact angle” is a measure of the wettability of a surface or material. In one aspect, the contact angle is the angle formed between the surface and a liquid droplet. Generally, when water is used as the liquid, the smaller the contact angle (less than 90°), the more hydrophilic or wettable the surface; and conversely, the larger the contact angle (greater than 90°), the more hydrophobic the surface.

As used herein, the “aspect ratio” is a ratio of the height of the microspot boundary, or the depth of the microspot itself, to another dimension of the microspot, which may be a length, width, or diameter of the microspot. This dimension is a measurement taken on a plane of the surface layer. Alternative or in addition, the heights, depths, or thicknesses referred to herein lie on a Z-axis of a Cartesian coordinate system, and the length, width, or diameter lie on an X-Y plane. For instance, a square or rectangular microspot may have a length and width, each ranging from about 1 μm-500 μm. Alternative, a circular microspot may have a diameter of ranging from about 1 μm-500 μm. The height of the microspot boundary, or depth of the microspot, as well as the height or thickness of the surface layer may range from about 0.5 to 5 nm. Thus, the aspect ratio of the microspot may be less than 0.01, preferably less than 0.001, or more preferably, less than 0.0001, which is significantly lower than the aspect ratio of previous microarrays. It is to be understood that the present invention is not limited to the aforementioned aspect ratios and that other values may be appropriate. For example, in some embodiments, the aspect ratio of the microspot may be a maximum of 2, or a maximum of 1, or a maximum of 0.5, or a maximum of 0.1.

In some preferred embodiments, the height of the boundary is sufficient to minimize mass transport limitations. Without wishing to limit the present invention, the height of the boundary, relative to a combined height of the vesicle and linker, is such that the diffusion limitations are minimized. In some embodiments, the height of the boundary is less than 10% of the combined height of the vesicle and linker. In other embodiments, the height of the boundary is less than 5% of the combined height. In still other embodiments, the height of the boundary is less than 3%, or less than 2%, or less than 1% of a combined height of the vesicle and linker. Alternatively or in additional embodiments, the height of the boundary may be less than 60% or less than 50% of the combined height. In another embodiment, the height of the boundary may be less than 40%, less than 30%, less than 25%, or less than 20% of the combined height. As used herein, the combined height of the vesicle and linker is the height taken from the bottom surface of the microspot on which the linker is attached to the topmost of the lipid membrane of the vesicle. In some embodiments, this combined height may range from 100-500 nm. For instance, the combined height may be about 100-200 nm, or about 200-300 nm, or about 300-400 nm, or about 400-500 nm. In other embodiments, the combined height may be greater than 500 nm. For example, the combined height may be 500-900 nm. It is to be understood that the present invention is not limited to the aforementioned heights, and that other values may be appropriate.

As known by one of ordinary skill in the art, the term “amination” and its equivalents refer to a process in which an amine group is introduced into a molecule. For example, the vesicles of the present invention may be amidated so as to have amine functional groups.

Microarrays

Referring now to FIGS. 1A-1D, in some embodiments, the present invention features a method of producing a microarray (100) for detection of membrane-binding analytes. The method may comprise providing a base substrate (110), applying a surface layer (120) to a top surface of the base substrate, and imprinting the surface layer (120) with a microspot array using photolithography techniques. The microspot array may comprise a plurality of microspots (140), each microspot (130) having boundaries (135) formed by the surface layer (120) and a bottom surface formed by the top surface of the base substrate. Continuing on, the method may further comprise modifying the bottom surfaces of the microspots with a linker group (140), preparing a plurality of functionalized, stabilized vesicles (150), and depositing the vesicles (150) within the microspot array. In preferred embodiments, the vesicles (150) are immobilized by covalently attaching to the linker group (140), thereby forming the microarray (100).

In some embodiments, the photolithography techniques for imprinting the surface layer (120) with the microspot array may comprise applying a photoresist film to a top surface of the surface layer, applying over the photoresist film a patterned mask having a pattern of the microspot array, and exposing the patterned mask to ultraviolet (UV) light such that the UV light only passes through the pattern to contact the photoresist film, thereby transferring the pattern to the photoresist film. The procedure may further comprise developing the photoresist film such that areas of the photoresist film corresponding to the pattern is removed, thereby exposing areas of the surface layer corresponding to the pattern, etching the exposed areas of the surface layer corresponding to the pattern, thereby exposing the top surface of the base substrate and imprinting the surface layer (120) with the microspot array, and removing the remaining photoresist film. Photolithography is given as an example technique; however, other etching processes may be used to pattern and imprint the microspot array onto the base substrate (110).

In other embodiments, the step of modifying the bottom surfaces of the microspots (130) with a surface modifier comprises silanizing the bottom surfaces and attaching a reactive moiety to the silane groups. Thus, the silane group and reactive moiety form a linker group (140) to which each lipid vesicle can covalently attach.

According to another embodiment, the present invention may feature a stabilized microarray (100) for detection of membrane-binding analytes. The microarray (100) may comprise a base substrate (110), a surface layer (120) disposed on a top surface of the base substrate, and a plurality of vesicles (150). In preferred embodiments, the surface layer (120) may comprise a microspot array having a plurality of microspots (130). In some embodiments, the microspot array may be formed in the surface layer (120) via photolithography techniques. Each microspot (130) can have boundaries (135) formed by the surface layer (120) and a bottom surface formed by the top surface of the base substrate (110). Further still, the bottom surface may be surface-modified with linker groups (140). In other embodiments, the vesicles (150) may be disposed within the microspot array such that the vesicles (150) are immobilized by covalent attachment to the linker group (140). In some embodiments, the vesicles (150) may be functionalized, stabilized lipid vesicles.

In one embodiment, the base substrate (110) may be a glass substrate, such as a glass slide, glass cover slips, or a substrate having a glass surface. In another embodiment, the surface layer (120) may comprise PEG. The PEG layer may be a thin film or coating disposed on the base substrate (110), thereby forming a PEGylated substrate. PEG is but one example material that may be used in the microspot array. However, in other embodiments, the PEG layer may be replaced with any surface layer material that can minimize non-specific surface binding.

In some embodiments, the linker group (140) may comprise a silane moiety attached to the bottom surfaces of the microspots. The silane moiety may be derived from a functionalized silane. Without wishing to limit the present invention to a particular theory or mechanism, the functionalized silanes are effective for modifying the exposed surfaces of the base substrate with reactive functional groups. Non-limiting examples of functionalized silanes include epoxy silanes, aminosilanes such as APTS, or thiol-functionalize silanes such as MPTS. However, the surface modification may be other suitable or equivalent moieties obtained from sources that would be known to one of ordinary skill in the art.

In other embodiments, the linker group (140) may further comprise an amine-reactive sulfonate moiety or other reactive moiety. Without wishing to limit the present invention to a particular theory or mechanism, the reactive moiety can provide a covalent attachment point for the lipid bilayer of each vesicle (150). For example, the vesicles (150) participate in a reaction with the reactive moiety of the linker group, thus forming covalent bonds between the vesicle (150) and linker group (140). In some embodiments, the bottom surface may be sulfonate modified such that the bottom surface comprises sulfonate molecules. Examples of sulfonates that may be used in accordance with the present invention include, but are not limited to, 2,2,2-trifluoroethanesulfonyl chloride, alkyl p-toluenesulfonates (tosylates) and related compounds. Sulfonate-modification is but one example of surface modification. It is to be understood that the surface of the microparticle may be modified with any suitable molecule that can provide a covalent attachment point for the lipid bilayer of each vesicle (150).

In yet another embodiment, the linker group (140) may further comprise an amine or thiol functional group to which each vesicle (150) is covalently attached. In one embodiment, the amine functionality may be obtained from APTS. In another embodiment, the thiol functionality may be obtained from MPTS. However, the surface functionality may be any other suitable or equivalent moiety obtained from a source that would be known to one of ordinary skill in the art.

In further embodiments, each microspot (130) can have a length and width dimensions of L×W μm. In one embodiment, L and W can each range from 5 to 500 μm. For example, the microspot (130) may have dimensions of 300×300 μm. In another embodiment, the microspot (130) can have a height or depth of about 0.5-5 nm. Thus, the microspots (130) have a low aspect ratio. In other embodiments, the microspot array may comprise the plurality of microspots (130) in a matrix arrangement of m×n (i.e. number of rows×number of columns). In one embodiment, m and n can each range from 10-1000. For example, the microarray may comprise the microspots (130) in a 100×200 arrangement. It is to be understood that the present invention is not limited to the aforementioned dimensions and that other values may be appropriate.

Nanoshell Vesicles

In some embodiments, the vesicles (150) may be functionalized, stabilized lipid vesicles. The lipid vesicles may comprise a plurality of lipid monomers formed into a lipid bilayer and polymerized with stabilizing agents, and one or more receptors. Without wishing to limit the invention to a particular theory or mechanism, the stabilizing agents can provide a polymer scaffold for stabilizing the lipid vesicles. In some embodiments, the one or more receptors may be covalently attached to an outer surface of the lipid vesicle, or embedded within the lipid bilayer, or both.

In other embodiments, the step of preparing the functionalized, stabilized lipid vesicles may comprise providing a plurality of lipid monomers, forming the plurality of lipid monomers into a plurality of lipid vesicles, each lipid vesicle having a lipid bilayer, functionalizing the lipid vesicles, and polymerizing the functionalized, lipid vesicles with stabilizing agents that are effective for providing a polymer scaffold for stabilizing the lipid vesicles.

In one embodiment, the step of functionalizing the lipid vesicles may comprise covalently attaching receptors, such as proteins, small molecules, nucleic acid aptamers or DNA oligomers, to an outer surface of the lipid vesicles. For example, these surface-attached receptors may be covalently linked to the functional groups of the surface modifiers, such as the amine or thiol functional groups.

In another embodiment, the step of functionalizing the lipid vesicles may comprise embedding receptors into the lipid bilayer. Non-limiting examples of said receptors include membrane protein receptors, growth factor receptors, G-protein coupled receptors, ion channels, lipid-derived receptors, glycoprotein receptors, glycolipids, phospholipids, or a combination thereof.

In some embodiments, a diameter of the vesicle may range from about 100-500 nm. For example, the vesicle diameter may range from about 100-200 nm, or about 200-300 nm, or about 300-400 nm, or about 400-500 nm. In other embodiments, each microspot (130) can have a depth of about 0.5-5 nm. Alternatively or in addition, the depth of the microspot may range from about 0.5-1 nm, or about 1-2 nm, or about 2-3 nm, or about 3-4 nm, or about 4-5 nm. In still other embodiments, when the vesicles (150) are covalently attached to the linker group (140), the combined height from the surface layer to the topmost of the vesicle can range from about 100-900 nm. For instance, the combined height may range from about 100-400 nm, or about 200-500 nm, or about 300-600 nm, or about 400-700 nm, or about 500-800 nm, or about 500-900 nm. It is to be understood that the present invention is not limited to the aforementioned dimensions and that other values may be appropriate.

In one embodiment, the lipid monomers may comprise polymerizable lipid monomers. For example, the polymerizable lipid monomers may be sorbyl- or dienoyl-containing lipid monomers such as 1,2-bis(octadeca-2,4-dienoyl)-sn-glycero-3-phosphocholine or 1,2-bis[10-(2′,4′-hexadieoyloxy)decanoyl]-sn-glycero-2-phosphocholine.

In another embodiment, the lipid monomers may comprise non-polymerizable lipid monomers. Examples of said non-polymerizable lipid monomers include, but are not limited to, cell membrane fragments, 1,2-dioleoyl-sn-glycero-3-phosphocholine (DOPC), 1,2-diphytanoyl-sn-glycero-3-phosphocholine monomers, naturally occurring lipids, synthetic lipids, or combinations thereof.

In yet another embodiment, the lipid monomers may further comprise functionalized lipid monomers. As an example, the functionalized lipid monomers can be amine functionalized lipid monomers having an amine functionality configured to covalently attach to the surface modifier. In an exemplary embodiment, the amine-functionalized lipid monomers may comprise amino(polyethylene glycol) (NH₂—PEG).

In one embodiment, each polymerized, hydrophobic non-lipid monomer may comprise a methacrylate, a styrene, or a combination thereof, and a cross-linking agent. In another embodiment, the methacrylate may be an aliphatic methacrylate, or an aromatic methacrylate such as benzyl methacrylate or naphthyl methacrylate. In yet another embodiment, the cross-linking agent may be a dimethacrylate such as, for example, ethylene glycol dimethacrylate.

In some embodiments, the stabilizing agents may comprise polymerizable, hydrophobic non-lipid monomers. In other embodiments, the stabilizing agents may comprise a cross-linking agent and a photoinitiator. In one embodiment, the cross-linking agent is a dimethacrylate, such as ethylene glycol dimethacrylate. In another embodiment, the photoinitiator is a methacrylate. For example, the methacrylate may be an aliphatic methacrylate such as butylmethacrylate, or an aromatic methacrylate such as benzyl methacrylate or naphthyl methacrylate.

Since the present invention provides microarrays and methods of fabricating said micorarrays, it is another objective of the invention to provide for methods of using said microarrays.

According to further embodiments, the present invention may feature methods for detecting one or more membrane-binding analytes simultaneously. In some embodiments, said method may comprise providing any of microarrays (100) described herein. Further still, said microarrays (100) may be fabricated according to any of the methods previously described. In preferred embodiments, the vesicles (150) of the microarray may comprise one or more receptors, including but not limited to, membrane protein receptors, growth factor receptors, G-protein coupled receptors, ion channels, lipid-derived receptors, glycoprotein receptors, glycolipids, phospholipids, or a combination thereof.

In another embodiment, the method for detecting one or more membrane-binding analytes may further comprise providing an analyte solution comprising the one or more membrane-binding analytes, adding the analyte solution to the microarray (100), and performing a detection technique on the assay platform. In some embodiments, the one or more membrane-binding analytes are capable of binding to their target receptor from the one or more receptors, thereby forming an analyte-bound assay platform. Without wishing to limit the invention to a particular theory or mechanism, each analyte may be identified by the target receptor to which the analyte is bound.

In some embodiments, the step of adding the analyte solution to the microarray (100) may comprise submerging the microarray (100) in the analyte solution. In alternative embodiments, the step of adding the analyte solution to the microarray (100) may comprise flowing the analyte solution across a surface of the microarray (100). In other embodiments, the detection technique may be fluorescent imaging or mass spectrometry. In preferred embodiments, the microarray technique can provide an important tool for multiplexed detection of a wide range of membrane-interacting analytes, including the example of bacterial toxin detection presented herein.

### EXAMPLES

The following is a non-limiting example of fabricating a microarray functionalized with stabilized phospholipid nanoshells and implementing said microarray in cholera toxin B detection. Equivalents or substitutes are within the scope of the present invention.

Referring to FIG. 1A, an exemplary method of fabricating a microarray from a PEG-based substrate is described as follows.

Preparation of PEGylated Glass Substrates

Glass cover slips (1.5 mm thickness) were first sonicated in methanol for 15 minutes, then were treated with a mixture of methanol (95%, w/v) and hydrochloric acid (37%) (1:1 v/v) for 30 minutes at room temperature. The samples were then thoroughly washed with water, blown dry with nitrogen gas and briefly heated at 60° C. for 10 minutes to dry out all of water residues. A solution comprised of 0.2% PEG silane in toluene (with 0.8 mL of HCl (37%)/L) was sonicated for 10 minutes prior to adding the glass cover slips. Samples were then shaken for 4 hours at room temperature. Afterwards, samples were washed twice with toluene, ethanol, water, and blown dry with nitrogen. Samples were then baked overnight at 60° C. oven, and stored dry under ambient conditions.

Photolithography Patterning and Surface Chemistry

A schematic diagram for surface pattering of PEGylated substrate was demonstrated in FIG. 1A. The photoresist film (AZ3312) was spin coated onto PEGylated substrate at 500 rpm for 20 seconds and 1500 rpm for 40 seconds. The photoresist film was thermally treated at 120° C. for 90 seconds. Selected film areas were exposed for 40 seconds at 310 nm with a typical dose of 150 mJ/cm² using a quartz mask with a Karl-Suss MA-6 mask aligner facilitated with a mercury lamp (350 W). The film was post baked at 120° C. for 90 seconds then was developed using AZ 300MIF developer solution for 90 seconds, washed with deionized water and dried with nitrogen stream. The samples were then treated with oxygen plasma (750 mTorr, 300 W) for 10 minutes to remove the PEG layer exposed from the uncovered photoresist area. After oxygen plasma etching, samples were washed with water to partially remove the etched PEGs, then the photoresist was lifted off from the samples by immersing in acetone for 5 minutes. The slides were thoroughly wash with water and then baked at 120° C. for 5 minutes to remove all of the water residues. At this stage, the glass slide had patterns of exposed hydrophilic glass surrounded by a coating of PEG molecules.

To functionalize the exposed hydrophilic glass patterns, samples were treated with epoxy silane in a vaporization chamber for 1 hour under vacuum conditions and were annealed overnight in 60° C. oven to stabilize the silane structures. Epoxy-grafted samples were then heated in 10 mM H₂SO₄ at 90° C. for 1 hour to convert epoxy rings into diols. After that, the slides were washed with water, acetone and blown dry. Diol-modified samples were placed in a nitrogen-flushed reaction chamber and a solution of 60 ml acetone/260 μl dry pyridine/180 μl tresyl chloride was added to convert diols into amine-reactive sulfonate derivatives. The reaction mixture was incubated for 40 minutes at room temperature with gentle shaking. Then the resulting sulfonate modified-slides were washed with acetone, acetone:5 mM HCl=1:1 (v/v), and 1 mM HCl, twice each. Finally, the slides were washed with water and acetone, blown dry and stored in a container sealed with parafilm at 4° C. until use to prevent moisture contamination.

Chemically Stabilized Phospholipid Nanoshells

Unilamellar phospholipid vesicles were prepared using a film hydration method. Briefly, DOPC in chloroform (10 mg), GM1 ganglioside (0.5% mol) and DSPE-PEG(2000)-NH₂ (1.5% mol) were mixed in a glass vial. The solvent was evaporated with a stream of argon gas and lyophilized for a minimum of 4 hours. The resultant dried lipid mixture was then rehydrated in 1 ml degassed PBS buffer (10 mM, pH 7.4) and first warmed up in a 42° C. water bath for 15 minutes. Samples were then subjected to 10 freeze-thaw-vortex cycles in warm water (42° C.) and dry ice/iso-propanol (−77° C.), followed by 21 time-extrusion through a two-stacked 200 nm Nuclepore polycarbonate filters using a stainless steel extruder. For fluorescence encapsulation inside the lipid membrane, rhodamine dextran (2 mg/mL in PBS, M_{w} 40 kDa) was added directly into the dried lipid films and freeze-thawed and extruded as described. Separation of non-encapsulated materials was performed on a Sepharose CL-4B column and rehydration buffer as the eluent.

To stabilize the phospholipid nanoshells, lipid membrane copolymerization was performed as follows: 2.5 mL buffered solution of unilamellar extruded DOPC was mixed with the cross-linking agent EGDMA and the photoinitiator BMA at a mole ratio of 1:2 for [DOPC]/[EGDMA] and 10:1 for [DOPC]+[EGDMA]/[BMA]. The resultant mixture was flushed with argon gas to illuminate oxygen and was stirred overnight at room temperature to facilitate partitioning of the monomers and photoinitiators into the lipid bilayer. Then a solution of redox reagents comprised of NaHSO₃ and (NH₄)₂S₂O₈ (10 mg/mL each) was added, the resultant mixture was then thermopolymerized at 42° C. for 2 hours with constant stirring to ensure homogenous polymerization. Size distribution was measured using a dynamic light scattering (DLS) system. Vesicle morphology was characterized by Transmission Electron Microscopy (TEM) and the lipid bilayer was stained with 0.7% uranyl acetate prepared in distilled water. To test the stability of the polymerized SPN, Triton X-100 was added at a [Triton X-100]/[lipid] molar ratio of 10 and the stability during long-term storage was calculated by the maximum increase in fluorescent background of the vesicle solution under CCD camera. FIG. 1B illustrates a typical structure of the as-prepared multifunctionalized phospholipid vesicles with embedded polymer scaffold.

Nanoshell Immobilization and Cholera Toxin B Interaction on SPN Microarray

Atomic Force Microscopy (AFM):

AFM images were obtained in liquid tapping mode with blueDrive on a Cypher S AFM. Oxide-sharpened DNP-S tip A silicon nitride probes with nominal spring constant of 0.35 N/m, resonant frequency of 65 kHz and radius of 10 nm were used. Cantilevers were tuned to 17 kHz for measurements in buffer. Images were acquired at a scan rate of 1 Hz with 256 points per line. Image analysis was done using Asylum Research Version 15.

Fluorescent Microscopy:

Fluorescent images were collected on a Nikon Eclipse Quantum TE2000 with 4× objective, and a cooled CCD camera. Excitation was by a 100 W mercury lamp with a D540/25 nm band-pass filter reflected by a 565DCLP dichroic mirror, exposure time was 600 ms.

Cholera Toxin Detection:

As-prepared polymerized DOPC nanoshells were immobilized on sulfonated PEG-based microarrays at a concentration of 500 μg/mL in PBS buffer for 2 hours at room temperature. Microarrays were washed with PBS buffer prior to imaging.

For toxin binding analysis, in one embodiment, Alexa Fluor 488 (AF488) succinimidyl ester was conjugated to cholera toxin B. Protein sample was purified by Sephadex G-25 using PBS as the eluent, final protein concentration was determined by Bradford assay using BSA as a standard. For toxin binding analysis, a serial dilution of CTB-AF488 was applied onto defined blocks of the SPN-microarray using a custom-made humidified reaction chamber. After 1 h, a serial washing with PBS was applied to remove any unbound toxins, the SPN microarray was scanned in the presence of degassed PBS using fluorescent microscope, and images acquired by the cooled CCD camera. In a primary screening experiment, the array was incubated with a known concentration of CTB-AF488; if positive signals are obtained on 90% of the microspots, more detailed analysis such as dose response and competitive binding assay are carried out. To quantitatively analyze the CTB-GM1 interaction, various concentrations of CTB-AF488 (0-10 μg/mL) were applied on SPN-microarrays. The binding buffer used for all experiments was PBS 10 mM, pH 7.4, 0.01% BSA. Signals were recorded after subtracting the background. Statistical data was obtained using Origin Pro 9. Each data point of each curve is represented as the mean±standard deviation (S.D) where n=9 replicates, curve fit to this data was based upon Hill-Waud binding model. Competitive binding assay was used to determine the equilibrium dissociation constant.

The detection of CTB in shrimp extract was also performed. Briefly, 3 grams of shrimp bought from a local market was weighed and an equal mass of PBS buffer was added. The mixture was homogenized, centrifuged at 8000 rpm for 5 mins and the supernatant was collected, stored in fridge and used within 9 h. Unlabeled CTB (0-50 μg/mL) of was spiked into the shrimp extract and then loaded onto SPN-microarray.

Results

Photolithographic Fabrication of SPN Microarray

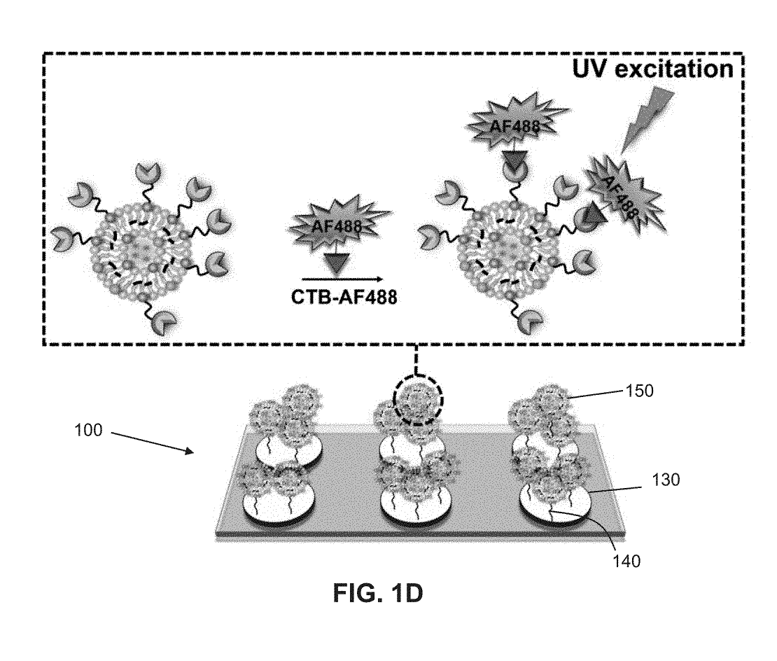

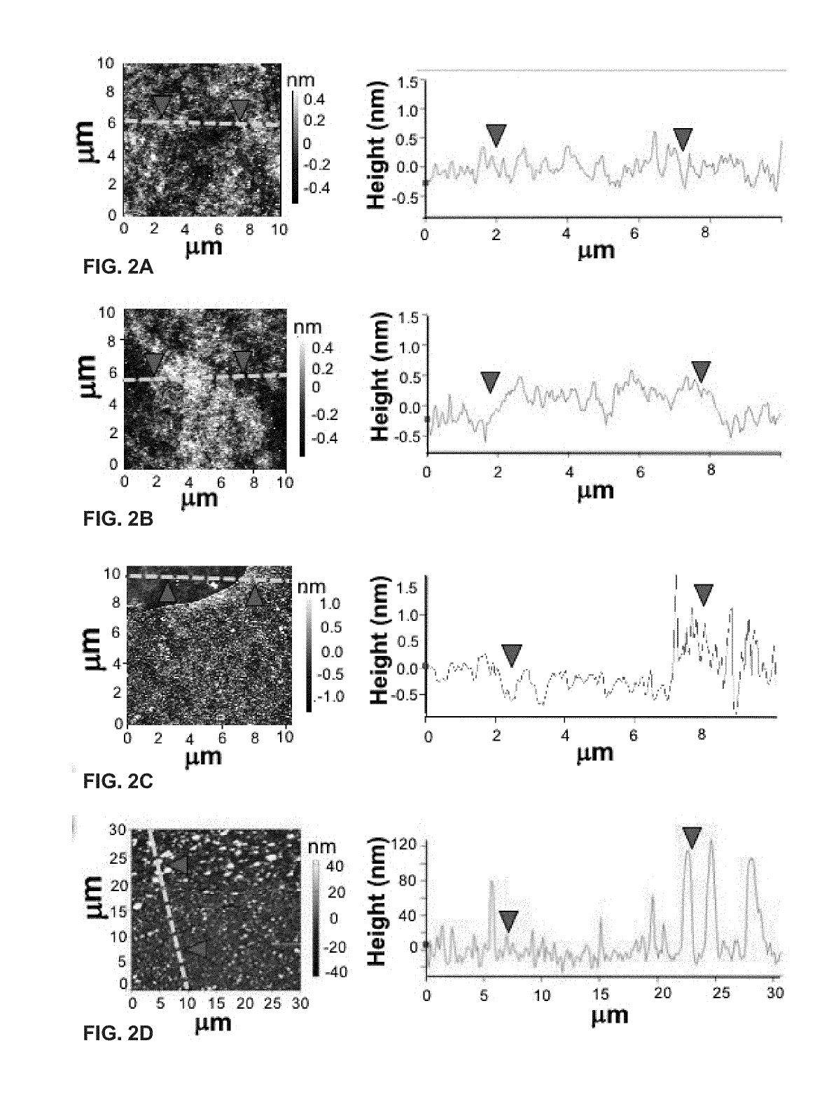

Atomic force microscope (AFM) imaging under aqueous conditions was employed for direct measurement and characterization of the SPN-microarray during step-by-step fabrication procedure, as shown in FIGS. 2A-2D. For glass cleaning, a mixture of MeOH/HCl (1:1 v/v) was used, and the cleaned, dried glass was treated with PEG-silane solution to prepare a substrate for photolithography. Without wishing to limit the present invention, the PEG-based substrate was used in soft photolithography due to its biocompatibility, hydrophilicity, and high resistance to protein adsorption and cell adhesion. Since traditional high aspect ratio-barriers of PEG hydrogel limit diffusional access to the nanoshell, a short PEG chain of 6-9 units was employed to minimize the diffusion interference. The PEGylated glass substrate exhibits a water contact angle of 36°, indicating good wettability.

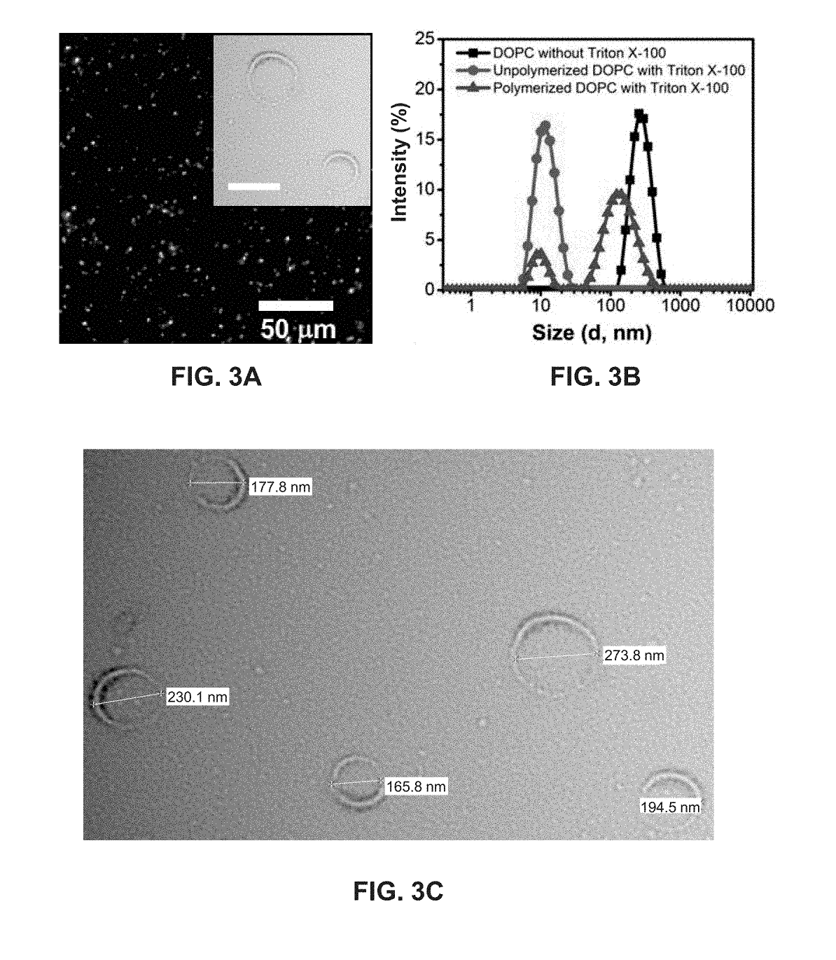

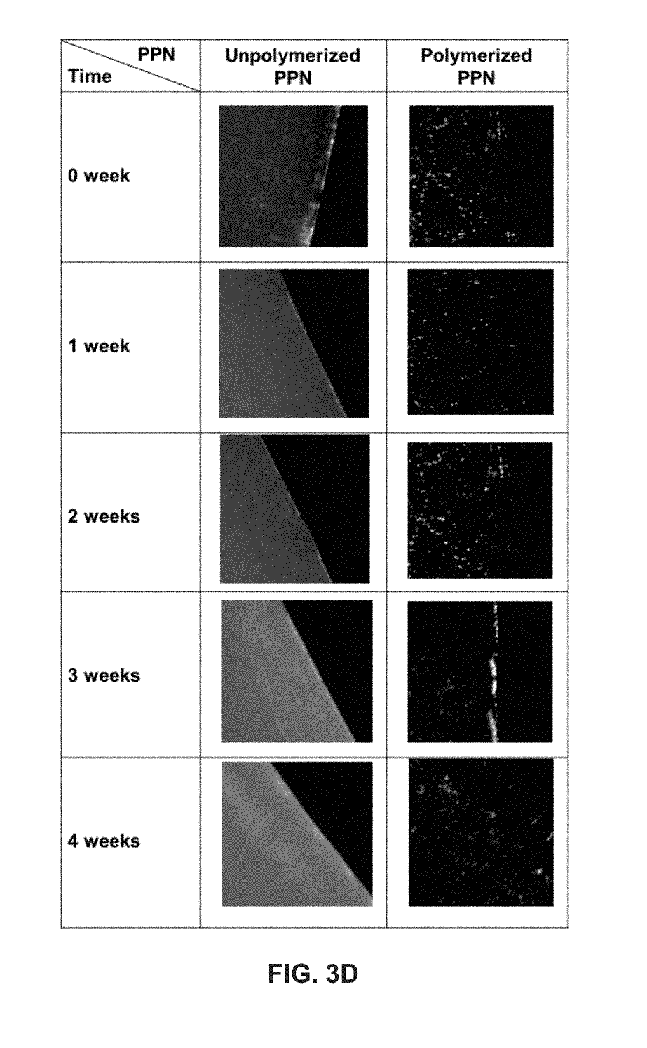

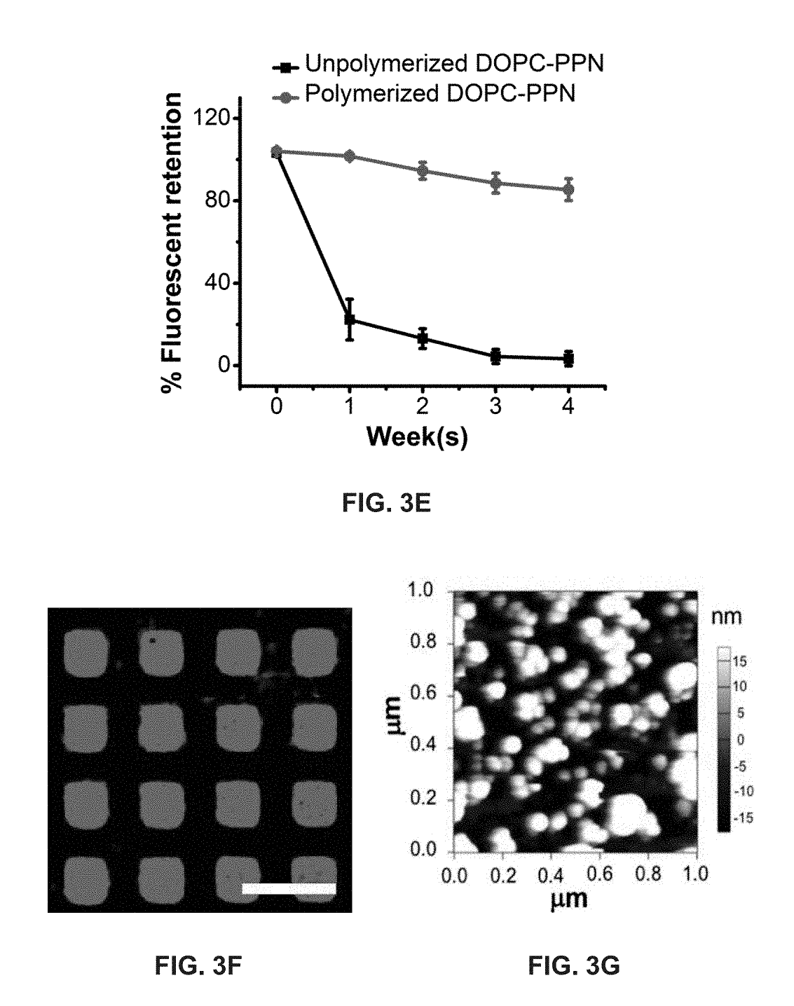

FIGS. 2A and 2B show 10×10 μm AFM image of glass surface after cleaning with MeOH/HCl solution and after PEG deposition, respectively, where no significant change in either height or morphology of the glass surface were observed. However, after etching the PEG glass with oxygen plasma in the presence of a photomask, a significant decrease in height was recorded across the arrayed microspot (FIG. 2C). The cross-section measurement shows a height difference of ca. 1.2 nm between the oxygen plasma treated-surface and the surrounded PEG layer. The contact angle measurement on PEG-substrate showed a large change in surface wettability, from 36° to <10° before and after plasma treatment, the contact angle was decreased significantly due to the increase of OH groups. The plasma treated-glass substrate was then immediately subjected to vapor silanization process where the silane solution was evaporated and introduced into the microspots by applying an external heat input of 120° C. in vacuum condition. Because the silane molecules formed on glass substrate by vapor silanization is relatively labile, the silanized surface was annealed at 60° C. for 3 h to form a stable polymerized network prior to sulfonate modification. Experiment results showed that a thermal curing at 60° C. prior to subsequent modification steps significantly increased the stability of the silanized network, which is critically important in this process. Non-specific modification outside the plasma treated-microspots was completely suppressed by the PEG layer. At the end of the process, the sulfonated microarray allows covalent immobilization of aminated SPNs which was encapsulated with Texas Red dextrans for fluorescent imaging confirmation (FIG. 3C). FIG. 2D shows 30×30 μm AFM image of the intersection between SPN immobilized-microspot and surrounding PEG layer. A clear boundary across the sampled intersection corresponding to a significant increase in height was observed (dashed yellow line). The immobilized SPNs were approx. 100 nm higher than the surrounding PEG molecules. Non-specific adsorption of SPNs on the PEG layer was negligible compared to the immobilized SPNs and did not affect the latter application. The sulfonate derivatized-array was active up to 45 day-storage at 4° C. in a desiccator chamber, making the developed approach attractive for other applications in sensor devices. The developed array fabrication process was then applied for the GM1 receptor functionalized SPN.

Stabilization of Phospholipid Nanoshell Functionalized with GM1 Ganglioside Membrane Receptor

In an exemplary embodiment, FIG. 1B illustrates the configuration of the GM1 ganglioside membrane receptor co-functionalized aminated-SPN (denoted as GM1-SPN). The lipid compositions in the SPN structure are completely tunable to control the surface architectures. For example, amine lipid was functionalized in the lipid bilayer to facilitate covalent immobilization onto sulfonate modified microarray. GM1 ganglioside membrane receptor was employed to demonstrate an example of membrane recognition from the binding of cholera toxin-B subunit (produced by Vibrio cholerae) to the ganglioside. Following preparation, hydrophobic crosslinking agents EGDMA and BMA was added into unilamellar suspension where they partition into lamellar of the hydrophobic lipid bilayer, forming polymer scaffold to stabilize the spherical structure of multifunctionalized SPN. For better visualization and characterization of the SPNs, Texas Red-conjugated dextran (MW 10 kDa) was encapsulated inside the SPN aqueous core during synthesis. FIG. 3A shows epifluorescent images of the SPN dispersed in PBS, appearing as bright spots due to fluorescence from encapsulated Texas Red-dextran. The inset picture shows a TEM image of spherical GM1-SPN structures in which the unilamellar lipid bilayer was revealed by uranyl acetate staining.

Referring to FIG. 3B, the mean diameter of the GM1-SPN was about 250±10 nm with polydispersity index 0.068, which is in agreement with diameters measured from TEM images as shown in FIG. 3C. The concentration of the polymerized scaffold formed inside the SPN bilayer leaflet can be varied in accordance with the desirable fluidity and the compatibility with the lipid components. To evaluate the enhanced stability of GM1-SPN structure embedded with polymer scaffold, the samples were treated with Triton X-100 at a 10-molar ratio of [Triton X-100]/[lipid]. The results showed that GM1-SPN with polymer scaffold exhibited insignificant change in diameter whereas self-assembled SPN dissolved into mixed micelles with a mean diameter of below 10 nm. As shown in FIGS. 3D-3E, fluorescent retention experiment showed that SPN supported with polymer scaffold retained approx. 90% of Texas Red fluorescence after 4 week-storage (at 4° C., in PBS buffer flushed with nitrogen), suggesting that polymer scaffold sustained the vesical configuration by providing a physical support to restrict vesicle fusion. On the other hand, self-assembled SPN maintained only 20% of encapsulated Texas red dextran with a dramatic fluorescent leakage after a week-storage.

Without wishing to be bound to a particular theory or mechanism, a key advantage of using an SPN structure is that its spherical architecture provides high volume ratio which is fundamentally similar to that of a natural cell membrane. FIG. 3F shows the false-color fluorescent image of SPNs immobilized on the microarray where bright red fluorescence from encapsulated Texas Red was recorded. FIG. 3G demonstrates 1×1 μm AFM image of SPN immobilized on microarray, where the configuration of immobilized SPNs was clearly revealed. Because self-assemble lipid vesicle ruptures almost immediately upon adsorbing on a surface, its application in microchip still remains extremely challenging. However, the present invention provides a polymer scaffold embedded within SPN that not only increases the SPN stability but also preserves the nanoshell configuration upon contacting the microarray substrate. According to AFM data, the spherical structure of the lipid nanoshells remained intact, even through multiple air-water interface withdrawals, without significant morphological deformation. Combined, these data proved the important role of polymer scaffold in improving the stability of SPN structures. The formation of polymerized scaffold in lipid bilayer leaflet offers a broadly accessible approach for preparing highly stabilized multifunctionalized lipid nanoshell. Additionally, the mild polymerization condition described herein can be amenable to a wide range of receptor and membrane protein-functionalized platforms. The as-prepared GM1-SPN microarray was then used for the detection of cholera toxin B.

Cholera Toxin Binding Assay

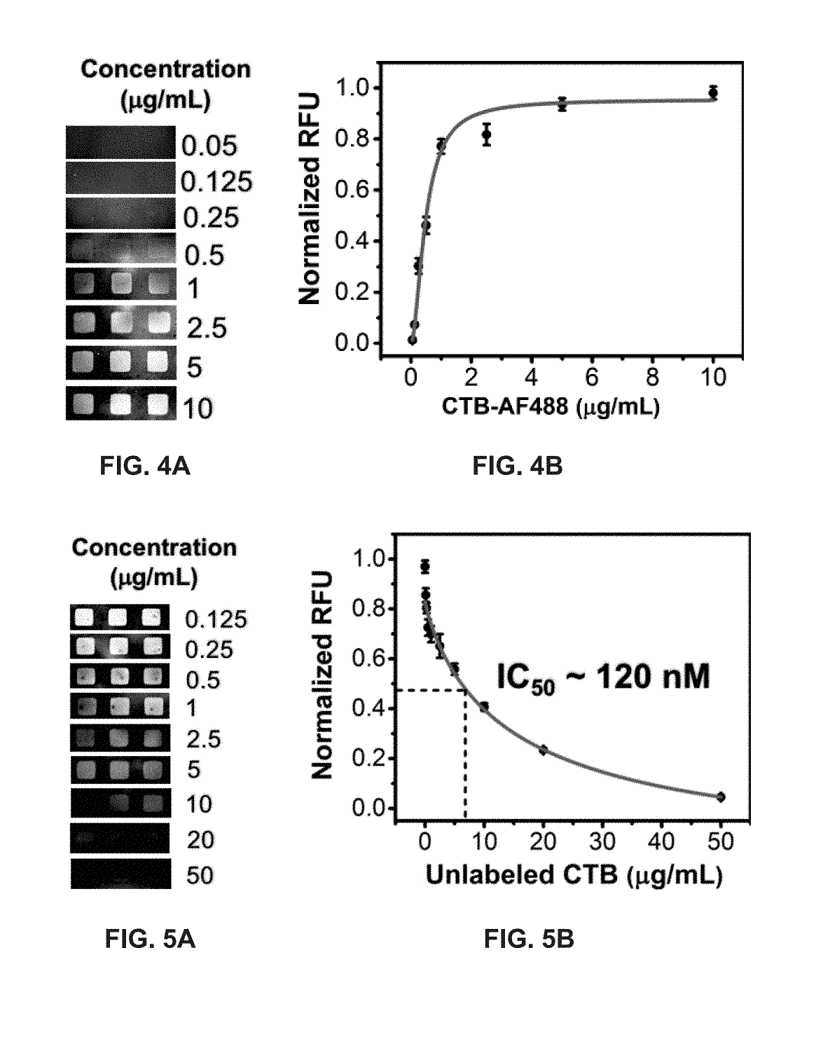

Referring to FIG. 1C, to demonstrate the biomolecule recognition of the GM1-SPN microarray described here, the prepared microarray was incubated in appropriate concentrations of cholera toxin B-subunit (CTB) fluorescently labeled with Alexa Fluor 488 (CTB-AF488). GM1 transmembrane element, which is found on the surface of epithelial cells, acts as a receptor to bind to cholera toxin B (CTB) secreted by the foodborne pathogen V. cholerae. Once CTB binds to GM1 receptors present on the surface of the lipid biomembrane, the toxin can trigger a fluorescent change in the arrayed microspots according to the amount of receptor-ligand interactions. After multiple washings with buffer to remove unbound toxins, the microarray was scanned using a fluorescent microscope. FIG. 4A shows fluorescent images of three replicate microspots treated with increasing concentration of CTB-AF488 ranging from 0 to 10 μg/mL, with the corresponding plot of the dose-response challenge shown in FIG. 4B. Strong binding of CTB-AF488 to GM1-DOPC microarray was observed and the dependency of the signal obtained on the concentration of CTB-AF488 followed the expected trend. The amount of binding is linear at 50-1000 ng/mL, specific binding was observed at a concentration as low as 15 ng/mL of CTB-AF488.

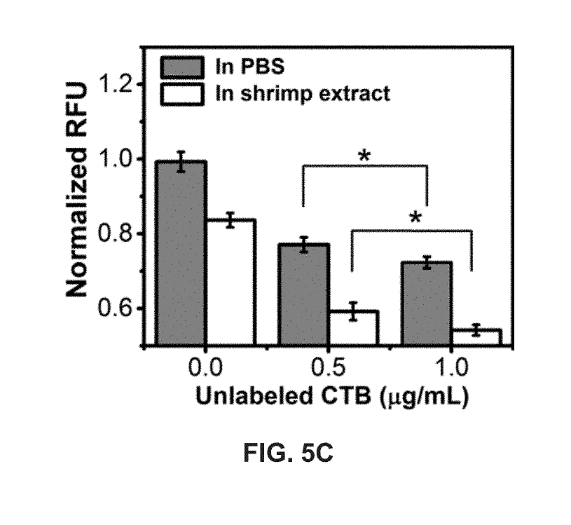

The binding of cholera toxin to ganglioside is multivalent and the binding affinity is dependent on the valency of the interaction. Therefore, the binding cannot be characterized by a single dissociation constant. For that reason, the affinity of CTB in terms of a IC₅₀ value was measured. To estimate this IC₅₀, a competitive binding assay was performed in which GM1-DOPC microarrays were treated with increasing concentration of non-labeled CTB at a fixed concentration of CTB-AF488 (2.5 μg/mL). The results in FIG. 5A showed the decreasing fluorescent intensities of CTB-AF488 when non-labeled CTB concentrations increased. From the inhibition profile shown in FIG. 5B, the IC₅₀ was estimated to be about 7 μg/mL (˜120 nM) and the detection limit for the unlabeled toxins is approximately 25 ng/mL, which is more sensitive than those obtained using soluble monosaccharide and immobilized GM1 receptors. A dissociation constant, Kᵢ, for CTB was estimated to be 5×10⁻⁹ M using the IC₅₀ value and the formula:

$${K}_{i} = \frac{{IC}_{50}}{1 + \frac{L}{{K}_{L}}} ,$$

where Kᵢ is the equilibrium dissociation constant for the inhibitor, L is the concentration of CTB-AF488, and K_{L} is the equilibrium dissociation constant for fluorescently labeled-CTB (˜2 nM). The proposed GM1-SPN microarray platform allows sensitive monitoring of cholera toxin B via fluorescent scanning in aqueous condition, proven by the dissociation constant in the nanomolar range and lower detection limit than previously reported.

Additionally, an experiment for cholera toxin detection in biological matrix (shrimp extract) was also demonstrated. Briefly, 0-1 μg/ml of unlabeled CTB was spiked into a prepared shrimp matrix in the presence of 2.5 μg/ml of CTB-AF488 and the sample was incubated with GM1-SPN microarray for 1 hr, followed by a serial of washings and subsequent fluorescent scanning. Despite the presence of large excess of other proteins in shrimp extract, the decrease in fluorescent intensity according to competitive binding of unlabeled CTB on microarray was observed (FIG. 5C), the recovery of fluorescent detection in shrimp extract was roughly 82% compared to detection in PBS with minimal sample preparation. This result demonstrated that CTB can be selectively captured on GM1-SPN microarray when it is present in a complex biological matrix. As such, the present invention provides an ultrasensitive, high-throughput screening method for cholera toxin and toxin inhibitor.

Microarrays of cell membrane components, such as stabilized-biomimetic membranes, provide an attractive platform for studying the fundamental aspects of cellular signaling and cell functions. The present invention provides a facile methodology to fabricate an array of chemically stabilized, biofunctionalized phospholipid nanoshells and has been demonstrated in the application of bacterial toxin detection. Chemically stabilized phospholipid nanoshells with excellent stability and ease of surface modification are an attractive platform for further applications in cellular membrane research ranging from cellular study to the detection of pathogens. The use of photolithography on a PEG-based substrate is an effective technique for assay development and the use of polymer scaffold-embedded lipid bilayer was found to be critical for enhancing the physical stability of the spherical lipid architecture, while also providing sufficient lateral movement for receptor-ligand binding.

As used herein, the term “about” refers to plus or minus 10% of the referenced number.

Various modifications of the invention, in addition to those described herein, will be apparent to those skilled in the art from the foregoing description. Such modifications are also intended to fall within the scope of the appended claims. Each reference cited in the present application is incorporated herein by reference in its entirety.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting of”, and as such the written description requirement for claiming one or more embodiments of the present invention using the phrase “consisting of” is met.

The reference numbers recited in the below claims are solely for ease of examination of this patent application, and are exemplary, and are not intended in any way to limit the scope of the claims to the particular features having the corresponding reference numbers in the drawings.

### REFERENCES

L. D. Wang, A. J. Wagers, Nat. Rev. Mol. Cell Biol., 2011, 12, 643.
K. P. Mellwig, M. Farr, J. Diekmann, D. Horstkotte, F. van Buuren, Dtsch. Med. Wochenschr. 2016, 141, 878.
S. M. Sugden, M. G. Bego, T. N. Pham, E. A. Cohen, Viruses. 2016, 8, 67.
A. Gonzalez-Barriga, J. Kranzen, H. J. Croes, S. Bijl, W. J. van den Broek, I. D. van Kessel, B. G. van Engelen, J. C. van Deutekom, B. Wieringa, S. A. Mulders, D. G. Wansink, PLoS One. 2015, 10, e0121556.
A. S. Hauser, S. Chavali, I. Masuho, L. J. Jahn, K. A. Martemyanov, D. E. Gloriam, M. M. Babu, Cell, 2018, 172, 41.
A. S. Hauser, M. M. Attwood, M. Rask-Andersen, H. B. Schioth, D. E. Gloriam, Nat. Rev. Drug Discov., 2017, 16, 829.
K. Dalal, C. S. Chan, S. G. Sligar, F. Duong, Proc. Natl. Acad. Sci. USA, 2012, 109, 4104.
H. A. Khamici, L. J. Brown, K. R. Hossain, L. H. Amanda, A. A. Sinclair-Burton, J. P. M. Ng, E. L. Daniel, J. E. Hare, B. A. Cornell, P. M. G. Curmi, M. W. Davey, S. M. Valenzuela, PLoS ONE. 2015, 10: e115699.
C. Peetla, A. Stine, V. Labhasetwar, Mol. Pharmaceutics, 2009, 6, 1264.
P. V. Escriba, J. M. Gonzalez-Ros, F. M. Goni, P. K. Kinnunen, L. Vigh, L. Sanchez-Magraner, A. M. Fernandez, X. Busquets, I. Horvath, G. Barcelo-Coblijn, J. Cell Mol. Med. 2008, 12, 829.
H. H. Shen, T. Lithgow, L. Martin, Int. J. Mol. Sci, 2013, 14, 1589.
S. Moreno-Flores, Biochim Biophys Acta, 2016, 1858, 793.
S. S. Hinman, C. J. Ruiz, G. Drakakaki, T. E. Wilkop, Q. Cheng, ACS Appl. Mater. Interfaces. 2015, 7, 17122.
E. B. Watkins, C. E. Miller, W. P. Liao, T. L. Kuhl, ACS Nano, 2014, 8, 3181.
H. A. Khamici, L. J. Brown, K. R. Hossain, L. H. Amanda, A. A. Sinclair-Burton, J. P. M. Ng, E. L. Daniel, J. E. Hare, B. A. Cornell, P. M. G. Curmi, M. W. Davey, S. M. Valenzuela, PLoS ONE. 2015, 10: e115699.
S. S. Hinman, C. J. Ruiz, G. Drakakaki, T. E. Wilkop, Q. Cheng, ACS Appl. Mater. Interfaces. 2015, 7, 17122.
F. W. Stetter, T. Hugel, Biophys. J. 2013, 104, 1049.
R. Molinaro, C. Corbo, J. O. Martinez, F. Taraballi, M. Evangelopoulos, S. Minardi, I. K. Yazdi, P. Zhao, E. De Rosa, M. B. Sherman, A. De Vita, N. E. T. Furman, X. Wang, A. Parodi, E. Tasciotti, Nat. Mater. 2016, doi: 10.1038/NMAT4644.
A. E. Saliba, I. Vonkova, A. C. Gavin, Nat. Rev. Mol. Cell Biol., 2015, 16, 753.
M. Tanaka, E. Sackmann, Nature, 2005, 437, 656.
S. P. Rajapaksha, X. Wang, H. P. Lu, Anal. Chem., 2013, 85, 8951.
E. Diamanti, E. Gutierrez-Pineda, N. Politakos, P. Andreozzi, M. J. Rodriguez-Presa, W. Knoll, O. Azzaroni, C. A. Gervasi and S. E. Moya, Soft Matter, 2017, 13, 8922.
Y. H. Chan, P. Lenz, S. G. Boxer, Proc. Natl. Acad. Sci. USA, 2007, 104, 18913.
A. E. Saliba, I. Vonkova, S. Deghou, S. Ceschia, C. Tischer, K. G. Kugler, P. Bork, J. Ellenberg, A. C. Gavin, Nat. Protoc., 2016, 11, 1021.
T. L. Williams, A. T. A. Jenkins, Langmuir. 2008, 130, 6438.
A. Chifen, R. Foerch, W. Knoll, H. Khor, P. J. Cameron, T. L. Williams, A. T. A. Jenkins, Langmuir. 2007, 23, 6294.
M. Yamada, H. Imaishi, K. Morigaki, Langmuir, 2013, 29, 6404.
J. J. Diaz-Mochon, G. Tourniaire, M. Bradley, Chem. Soc. Rev. 2007, 36, 449.
N. Vafai, T. W. Lowry, K. A. Wilson, M. W. Davidson, S. Lenhert, Nanofabrication, 2015, 2, 34.
A. E. Kusi-Appiah, N. Vafai, P. J. Cranfill, M. W. Davidson, S. Lenhert, Biomaterials, 2012, 33, 4187.
P. S. Cremer, S. G. Boxer. J. Phys. Chem. B. 1999, 103, 2554.
Y. Wang, G. T. Salazar, J. H. Pai, H. Shadpour, C. E. Sims, N. L. Allbritton, Lab Chip. 2008, 8, 734.
S. W. Han, S. Lee, J. Hong, E. Jang, T. Lee, W. Koh, Biosens. Bioelectrons. 2013, 15, 129.
H. Pan, Y. Xia, M. Qin, Y. Cao, W. Wang, Phys. Biol. 2015, 12, 045006.
J. U. Lee, A. H. Nguyen, S. J. Sim, Biosens. Bioelectrons. 2015, 74, 341.
C. R. Vista, A. C. P. Aguas, G. N. M. Ferreira, Appl. Surf. Sci, 2013, 286, 314.
A. Gang, G. Garbenet, L. D. Renner, L. Baraban, G. Cuniberti, RSC Adv, 2015, 5, 35631.
P. Larsson, Methods in Enzymology, 1984, 104, 212.
Z. Cheng, C. A. Aspinwall, Analyst, 2006, 131, 236.
E. Mansfield, E. E. Ross, C. A. Aspinwall, Anal. Chem. 2009, 79, 3135.
T. L. Williams, A. T. A. Jenkins, Langmuir. 2008, 130, 6438.
L. K. Bright, C. A. Baker, R. Branstrom, S. S. Saavedra, C. A. Aspinwall, ACS Biomater. Sci. Eng, 2015, 1, 955.
K. Dimitrievski, B. Kasemo, Langmuir, 2009, 25, 8865.
O. Teschke, Langmuir. 2002, 18, 6513.
A. Chifen, R. Foerch, W. Knoll, H. Khor, P. J. Cameron, T. L. Williams, A. T. A. Jenkins, Langmuir. 2007, 23, 6294.
M. Mammen, C. Seok-ki, G. M. Whitesides, Angew. Chem. Int. Ed. Engl. 1998, 37, 2755.
C. R. MacKenzie, T. Hirama, K. K. Lee, E. Altman, N. M. J. Young, Biol. Chem. 1997, 272, 5533.
Y. Fang, A. G. Frutos, J. Lahiri, Langmuir, 2003, 19, 1500.
G. M. Kuziemko, M. Stroh, R. C. Stevens, Biochemistry. 1996, 35, 6375.
J. M. Moran-Mirabal, J. B. Edel, G. D. Meyer, D. Throckmorton, A. K. Singh, H. G. Craighead, Biophys. J. 2005, 89, 296.
C. A. Rowe-Taitt, J. J. Cras, C. H. Patterson, J. P. Golden, F. S. Ligler. Anal. Biochem., 2003, 281, 123.
M. M. Ngundi, C. R. Taitt, S. A. McMurry, D. Kahne, F. S. Ligler, Biosens. Bioelectrons. 2006, 21, 1195.
Q. Cheng, S. Zhu, J. Song, N. Zhang, Analyst, 2004, 129, 309.
B. Liang, Y. Ju, J. R. Joubert, E. J. Kaleta, R. Lopez, I. W. Jones, H. K. Jr. Hall, S. N. Ratnayaka, V. H. Wysocki, S. S. Saavedra, Anal. Bioanal. Chem, 2015, 407, 2777.
A. Papra, N. Gadegaard, N. B. and Larsen, Langmuir, 2001, 17, 1457.

What is claimed is:

1. A microarray comprising a plurality of microspots and a plurality of vesicles, each vesicle tethered to a microspot of the plurality of microspots via a linker group, wherein said linker group is effective for enhancing mechanical stability of the microarray by covalently immobilizing said vesicle to the microspot.

2. The microarray of claim 1 further comprising a surface layer in which the microspots are formed therein, wherein the surface layer is effective for minimizing non-specific surface binding outside of the microspots.

3. The microarray of claim 2, wherein each microspot has a boundary formed by the surface layer, wherein a height of the boundary and the surface layer surrounding the microspot is less than 10% of a combined height of the vesicle and linker group.

4. The microarray of claim 1, wherein the linker group has a proximal end comprising a silane moiety attached to a bottom surface of the microspot, and a distal end covalently immobilizing said vesicle to the microspot.

5. The microarray of claim 1, wherein the vesicles comprise lipid monomers that are polymerizable lipid monomers, non-polymerizable lipid monomers, functionalized lipid monomers, or a combination thereof.

6. The microarray of claim 5, wherein the vesicles further comprise a polymer scaffold for stabilizing the vesicles.

7. The microarray of claim 5, wherein the vesicles are functionalized with receptors covalently attached to an outer surface of the vesicles or embedded into a lipid structure of the vesicles.

8. The microarray of claim 7, wherein the receptors are membrane protein receptors, growth factor receptors, G-protein coupled receptors, ion channels, lipid-derived receptors, glycoprotein receptors, glycolipids, phospholipids, or a combination thereof.

9. A microarray platform comprising a plurality of microspots modified with linker groups, wherein the linker groups are effective for enhancing mechanical stability of the microarray platform when said linker groups covalently immobilize vesicles to the microspots.

10. The microarray platform of claim 9, wherein each linker group has a proximal end comprising a silane moiety attached to a bottom surface of the microspot, and a distal end comprising a reactive functional group.

11. The microarray platform of claim 9 further comprising a surface layer in which the microspots are disposed therein, wherein the surface layer is effective for minimizing non-specific surface binding outside of the microspots.

12. The microarray platform of claim 11, wherein each microspot has a boundary formed by the surface layer, wherein an aspect ratio of the microspot is less than 0.01.

13. A method of preparing a microarray, said method comprising forming an array of microspots, and tethering a vesicle to an interior of the microspots via a linker group, thereby enhancing mechanical stability of the microarray by covalently immobilizing said vesicle to the microspot.

14. The method of claim 13, wherein the array of microspots are formed in a surface layer, wherein the surface layer is effective for minimizing non-specific surface binding outside of the microspots.

15. The method of claim 14, wherein each microspot has a boundary formed by the surface layer, wherein a height of the boundary and the surface layer surrounding the microspot is less than 10% of a combined height of the vesicle and linker group.

16. The method of claim 13, wherein the linker group has a proximal end comprising a silane moiety attached to a bottom surface of the microspot, and a distal end comprising a reactive functional group that reacts with the vesicle, thereby covalently immobilizing said vesicle to the microspot.

17. The method of claim 13, wherein the vesicles comprise lipid monomers that are polymerizable lipid monomers, non-polymerizable lipid monomers, functionalized lipid monomers, or a combination thereof.

18. The method of claim 17, wherein the vesicles further comprise a polymer scaffold for stabilizing the vesicles.

19. The method of claim 17, wherein the vesicles are functionalized with receptors covalently attached to an outer surface of the vesicles or embedded into a lipid structure of the vesicles.

20. The method of claim 19, wherein the receptors are membrane protein receptors, growth factor receptors, G-protein coupled receptors, ion channels, lipid-derived receptors, glycoprotein receptors, glycolipids, phospholipids, or a combination thereof.
